(19) United States
(12) Patent Application Publication
(10) Pub. No.: US 2010/0152594 A1
(43) Pub. Date: Jun. 17, 2010

(54) NON-INVASIVE DEVICE NADI TARANGINI USEFUL FOR QUANTITAVE DETECTION OF ARTERIAL NADI PULSE WAVEFORM

(76) Inventors: Ashok Bhat, Maharashtra (IN); Aniruddha Joshi, Maharashtra (IN); Anand Kulkarni, Maharashtra (IN); Bhaskar Kulkarni, Maharashtra (IN); Valadi Jayaraman, Maharashtra (IN); Sharat Chandran, Maharashtra (IN)

Correspondence Address: ABELMAN, FRAYNE & SCHWAB, 666 THIRD AVENUE, 10TH FLOOR, NEW YORK, NY, 10017, US

(21) Appl. No.: 12/733,153

(22) PCT Filed: Aug. 7, 2008
(86) PCT No.: PCT/IN2008/000494

§ 371 Date: Feb. 5, 2010

(30) Foreign Application Priority Data
Aug. 7, 2007 (IN) 1675/DEL/2007

(51) Int. Cl. A61B 5/0245

(52) U.S. Cl. 600/501

(57) ABSTRACT

The present invention discloses the procedure for obtaining complete spectrum of the Nadi pulses, as a time series and capable of detecting the major types and the subtypes of the Nadi pulses. The device of this invention involves three diaphragm elements equipped with strain gauge, three transmitters cum amplifiers, and a digitizer for quantifying analog signal. The system acquires the data with 12-bit accuracy with practically no electronic and/or external interfering noise. The pertaining proofs are given which clearly shows the capability of delivering the accurate spectrums, with repeatability of the pulses from the invented system. ‘Nadi-Nidan’ is a prominent method in Ayurveda (Ayurveda is a Sanskrit word derived from ‘Ayus’ and ‘vid’, meaning life and knowledge respectively. It is a holistic science encompassing mental, physical and spiritual health), which is known to dictate all the salient features of a human body. Nadi-Nidan is a specialty of ‘Vaidyas’ (Ayurvedic physicians) and hence the present system would enable the diagnosis accurately, quantitatively and independent of any human errors.

### FIELD OF THE INVENTION

The present invention relates to a non-invasive device Nadi Tarangini useful for quantitative detection of arterial nadi pulse waveform. More particularly, the present invention relates to an apparatus for obtaining the complete spectrum of the Nadi (arterial pulse) as a time series and application of advanced machine learning algorithms to identify the pulse patterns. According to the present invention, three diaphragm-based strain gauge elements are to be placed at the exact pick up positions (known as Vata, Pitta and Kapha positions) at the root of thumb on a hand wrist, which experience the pressure exerted by the radial artery and give equivalent electrical output. Each electrical output, coupled with the excitation of the strain gauge at the transmitter, is then digitized using a digitizer, having an interface with the personal computer at the USB port. This pressure is tiny in pressure units is captured in accurate, reproducible and noise-free waveforms to perform accurate diagnosis. A very small air gap is introduced between each of the sensing elements and the skin of person for capturing the exact values. The typical physiological properties such as rhythm, self-similar nature, and chaotic nature present in the pulse are extracted using rigorous machine learning algorithms. Subsequently the six pulse waveforms obtained through our invention (three waveforms on each hand) are classified as various types and sub-types of nadi patterns, primarily defined in the Ayurvedic literature.

The system of the present invention is intended to eliminate all the human errors in the Nadi-Nidan performed manually by Ayurvedic practitioner and the diagnostics could be performed based on accurate and quantitative information. The invention could also eliminate any subjectiveness in the diagnostics.

### BACKGROUND AND PRIOR ART

Ayurveda (Indian Traditional Medical science) believes that the function of entire human body is governed by three humors: Vata, Pitta and Kapha, collectively called as Tridosha. The equilibrium of these three doshas maintains the proper functioning of every aspect of physiology. Any imbalance in the proportion causes a disorder. The imbalance causes the vessels carrying the blood to contract or expand with respect to its normal position. This contraction/expansion of vessels results in modulation of blood flow, which is called as Nadi. In brief, Nadi dictates the mode of blood circulation, which no doubt is governed by the physiological state of the individual. This makes Nadi-Nidan [meaning diagnosing a disease by sensing the blood flow] as a first step and in most cases the only diagnostic tool for patient diagnosis, according to Ayurveda.

There are around 74000 locations in human body where the Nadi pulses can be obtained, out of which only two positions are in proximity. However, the standard position according to Ayurvedic practitioners is at the root of thumb on the wrist as shown in FIG. 1. The three fingers of an Ayurvedic practitioner's hand, 1 in FIG. 1, namely the index finger, 2 in FIG. 1, the middle finger, 3 in FIG. 1, and the ring finger, 4 in FIG. 1, are placed at the root of thumb of a patient's hand, 5 in FIG. 1, and the pulses can be sensed at the fingertip. Each finger senses Vata prakriti, 6 in FIG. 1, Pitta prakriti, 7 in FIG. 1, and Kapha prakriti, 8 in FIG. 1, respectively. The general characteristics of these pulses are given in the Table 1.

All the pulses sensed at the fingertip have been traditionally further classified as Sukshma, Tikshna, Kathina and Sama as major types and Vegavati (fast), Manda (slow), Khol (deep) as few of the subtypes and their combinations. The above classification is mainly based on the excursion and pulse movements. The nature of these pulses can be expressed in terms of the parameters such as frequency, depth, power, rhythm. All these parameters are sensed at the predetermined pick-up points on each of the fingertips. Any change in these characteristics represents the kind of disorder.

Some of the previous related references include U.S. Pat. No. 6,432,060, US 20031009105, U.S. Pat. No. 6,364,842, U.S. Pat. No. 5,623,933, U.S. Pat. No. 5,755,229, U.S. Pat. No. 5,832,924, U.S. Pat. No. 5,938,618, U.S. Pat. No. 6,155,983, U.S. Pat. No. 6,159,166, U.S. Pat. No. 6,261,235, U.S. Pat. No. 6,364,842, U.S. Pat. No. 6,767,329, U.S. Pat. No. 6,293,915, U.S. Pat. No. 6,730,040, U.S. Pat. No. 7,074,193, U.S. Pat. No. 7,192,402 and U.S. Pat. No. 7,195,596. Some of these approaches capture pulse waveforms from fingertips instead of wrist positions. Some approaches apply pressure on the position using compressed air to take the pulse, which changes the pulse reading. It is also difficult to tell if the Nadi obtained using these methods is complete or not.

The drawbacks of the hitherto reported prior art can be summarized as follows:
The above description itself dictates that the skill involved in the Nadi-Nidan comes through lot of practice and experience. Again, the information content is only qualitative and no quantitative conclusions can be drawn at the outset. Also there is subjectiveness in the reported processes.
In most of the previous attempts disclosed, the methodology involves application of some constant pressure (to obtain maximum amplitude) on the radial artery. But, it is known that Nadi-Nidan does not support any such external pressure on the artery, since it affects the blood circulation and hence the Nadi itself.
Further, for any diagnostic method, it is essential to know the completeness as well as inaccuracies (in the present case, the noise content of the waveform), which is not mentioned.
Most of the previous attempts disclosed just present the pulse waveforms or compute the pulse rate, but no further processing is presented towards diagnosis.

Thus, the inventors of the present invention realized that there exists a need to develop a system based on Ayurveda, which would overcome all these problems. Hence it was thought desirable to have a system, which can give Nadi pulses as a time-series data and yet simple to use. In the present disclosure all the limitations have been removed, hence the waveforms obtained from the present embodiment are used for diagnosis based on quantitative information. Further, all the major types and subtypes of the Nadi pulses have been identified, which supports the accuracy of the waveforms obtainable from the present disclosure.

The system of the present invention is intended to provide a convenient, inexpensive, painless, and noninvasive methodology to eliminate all the human errors in the Nadi-Nidan performed manually by Ayurvedic practitioner and the diagnostics could be performed based on accurate and quantitative information. The invention could also eliminate any subjectiveness in the diagnostics.

### OBJECTS OF THE INVENTION

Thus, the main object of the present invention is to provide a convenient, inexpensive, painless, and non-invasive Computer-aided device which will eliminate all the human errors in the Nadi-Nidan performed manually by Ayurvedic practitioner for diagnostics of disorders and human health parameters.

Another object of the invention is to provide a device which is easy-to-use and quick in response system, which removes the subjectiveness by performing based on accurate and quantitative information.

Yet another object of the invention is to provide a device which can give nadi pulses as a time-series data and yet simple to use.

Still another object of the invention is to provide a device wherein various machine learning algorithms have been applied on the nadi waveforms to classify the major types and subtypes of the nadi pulses, which supports the accuracy of the waveforms obtainable from the present disclosure.

### SUMMARY OF THE INVENTION

The methodology adapted in the present invention involves the placement of the pressure sensing element at the exact pick-up point of the fingertip, where nadi pulses are sensed and the analog pressure signal generated therein is digitized. The waveforms are then analyzed using modern machine learning techniques and are then classified into various types and sub-types of nadi defined in Ayuniedic literature.

The definitions of the terms used in the present invention are given here as under:
“Ayurveda”—Ayurveda is a Sanskrit word derived from two roots: ayur, which means life; and veda, which means knowledge. It has its roots in ancient vedic literature. Ayurveda, a system of diet, healing and health maintenance, is probably the oldest science of life, just like the science of Yoga.
“Nadi”—refers to pulse

The starting point for many people into the ancient scientific art of Ayurveda is the relationship of the three Doshas: Vata, Pitta and Kapha.

Ayurveda sees life as a harmonic flow, a dynamic balance of those three fundamental forces:
Vata (wind, air) [symbol] the principle of movement and impulse
Pitta (bile, fire) [symbol] the principle of assimilation and transformation
Kapha (mucus, water) [symbol] the principle of stability

These forces act in everyone. When they are in balance they bring well-being and health, in imbalance they lead to feeling unwell and later disease. Everybody is unique and Ayurveda respects this uniqueness. That is why there are individual constitution types, Doshas, in the body.

Out of the three basic forces seven categories of individuals can be formed:
1. Wind dominated individuals (vata)
2. Bile dominated individuals (pitta)
3. Mucus dominated individuals (kapha)
4. Wind and Bile dominated individuals (vata and pitta)
5. Wind and Mucus dominated individuals (vata and kapha)
6. Bile and mucus dominated individuals (pitta and kapha)
7. Wind, bile and mucus dominated individuals (vata and pitta and kapha in equal proportion)
“vegavati”—if the pulse rate is very high, and the movement is higher, then the pulse is detected as Vegavati pulse
“manda”—if the pulse rate is low with very less movements in Tidal and Dicrotic waves, then the pulse is detected as Mande pulse
“sukshma”—if the pulse has very low slopes with wide widths of Tidal and Dicrotic waves, then the pulse is detected as Sukshma pulse
“tikshna”—if the pulse has sharp slopes at the Percussion wave, then the pulse is detected as Tikshna pulse. It promotes sharpness and rapidity of comprehension.
“kathina”—if the shapes at the Tidal and Dicrotic waves look like equilateral triangle, then the pulse is detected as Kathina pulse. It increases strength, rigidity.
“sama”—if the pulse shows equivalent behaviour in all the three doshas, then the pulse is detected as Sama pulse.

As mentioned earlier, the Nadi pulses are sensed by the three fingertips of the Ayurvedic practitioner at the root of thumb on wrist, which actually measure the pressure exerted by the artery. This pressure is in fact very tiny (−0.00124 Pa to +0.00124 Pa) in pressure units. In the present invention similar methodology is used. Three pressure sensing elements (of pressure range of 3 inch H₂O to 5 inch H₂O) coupled with three transmitters (one for each one sensing element), which can amplify the electrical signal, are placed at the predetermined locations instead of the three fingertips, which generate three electrical signals proportional to the pressure experienced by the three pressure sensing elements. Each of the three electrical signals is then digitized using the digitizer, having an interface with the personal computer at the USB port. The data can be obtained on the computer for a predetermined length of time, for any change in the signal value, by using the data acquisition software, which controls the digitization as well. The minimum change in the signal, which can be measured, depends solely on the resolution of the ADC. The three such pulse data are stored against one time information on one hand. Similar pulse data are obtained for the second hand of the person.

The data obtained in this way is usually corrupted because of implicit and explicit electronic and electrical disturbances, called as noise, which modulates the information content. The noise level obtained in the present system developed is almost zero, after proper shielding. Hence the Nadi obtained is in pure form and any digital filtering on the signal obtained from the digitizer, of any kind, is not required.

Once the pulse data is stored on computer, Pitch Synchronous Wavelet Transform is applied on each pulse data series to extract the average properties. Then important physiological properties are computed using various feature extraction methods such as Fourier analysis, Chaos analysis, Variability analysis. Finally, types and sub-types of pulses are detected based on these parameters.

Accordingly, the present invention provides a non-invasive device Nadi Tarangini, useful for quantitative detection of arterial ‘nadi’ pulse waveform, wherein the said assembly comprising:
[a] at least three circuits of diaphragm based pressure sensors [1 in FIG. 2] placed side by side at the three predetermined exact pick up points on the wrist of a user [6, 7, 8 in FIG. 1] for sensing the ‘nadi’ pulses;
[b] at least one strip of neoprene [5 in FIG. 3] provided at the bottom of the said pressure sensors;
[c] the said strip provided with at least three holes [3 in FIG. 3] to introduce air gaps having thickness in the range of 1 to 5 mm for capturing the arterial pulsations;
[d] providing at least one transducer [1 in FIG. 2] corresponding to each of the said pressure sensor provided above along with a DC power source [4 in FIG. 4] for converting the pressure signal into an equivalent electrical signal;
[e] providing at least one digitizer [5 in FIG. 4] for converting the electrical signal obtained in step [d] above into digital form, using at least one Analog to Digital Converter (ADC) [5 in FIG. 4], along with a shielding arrangement [7 in FIG. 5] for minimizing the noise;
[f] providing a computing device [7 in FIG. 2] connected to the said digitizer for obtaining the visual display of the pulse pressure waveform.

### BRIEF DESCRIPTION OF THE ACCOMPANYING DRAWINGS

FIG. 1 shows the positioning of the fingertips of an Ayurvedic practitioner on patient's hand for sensing the pulse at three positions Vata, Pitta and Kapha.

FIG. 2 provides the schematic drawing of the present invention.

FIG. 3 shows the arrangement of neoprene sheet to introduce air gap between sensors and the patient's skin.

FIG. 4 is the electrical line diagram according to the present invention.

FIG. 5 is the circuit diagram for one of the sensing elements of the system according to the present invention (same circuitry is done for other two sensors).

FIG. 6 shows a sample pulse data for small duration from our database for three pick-up positions.

FIG. 7 shows a sample dosha waveform (of three) indicating the important time domain features.

FIG. 8 shows an example of Vegavati pulse

FIG. 9 shows an example of Manda pulse

FIG. 10 describes the steps involved in computation of average values to capture the essence of pulse data series using Pitch Synchronous Wavelet Transform (PSWT)

FIG. 11 shows an example of Tikshna Nadi

FIG. 12 shows an example of Kathina Nadi

FIG. 13 shows an example of Sama Nadi

FIG. 14 shows an example of Sukshma Nadi

FIG. 15 shows the variations in multifractal spectra of vata data series of persons in three age-groups.

FIG. 16 shows a sample arrhythmic pulse where every third beat is missing, and is captured by the variability of pulse intervals.

FIG. 17 shows the comparison between normal and fever pitta pulse through recurrence plot analysis.

FIG. 18 displays a flowchart indicating important steps in our approach of diagnosing a patient using data from our embodiment using rigorous machine learning algorithms.

FIG. 19 shows an example of pulse of person 32 at three predefined positions vata, pitta and kapha.

FIG. 20 shows and example of Vata pulse of person 32 for 1 minute.

FIG. 21 shows and example of Fourier transform of the vata pulse of person 32.

FIG. 22 shows an example of the detected peaks of vata pulse of person 32.

FIG. 23 shows an example of folding the vata pulse of person 32, so that all the peaks are together.

FIG. 24 shows and example of average vata pulse of person 32 showing the essence of the entire time series.

FIG. 25 shows an example of the multifractal spectrum of vata pulse of person 32.

FIG. 26 shows an example of the pulse rate variability indicating the time differences between the peaks of vata pulse of person 32.

FIG. 27 shows an example of the recurrence plot of vata pulse of person 32.

### DETAILED DESCRIPTION OF THE INVENTION

Time series analysis and Machine learning are useful tools to understand the underlying dynamics of the physiological system. In general, a time-series can be obtained by digitizing the analog signal from the pressure sensing element and the transducer, at the desired sampling rate and for desired time, by using a digitizer (analog to digital converter, ADC). ADC has an interface with personal computer (PC) which can transfer and store the data series, called as time series, on the disk. The time series obtained by this way can then be analyzed using various machine learning algorithms to extract the dynamic features of the underlying system. A similar methodology is adapted in the present invention to acquire the Nadi pulses quantitatively.

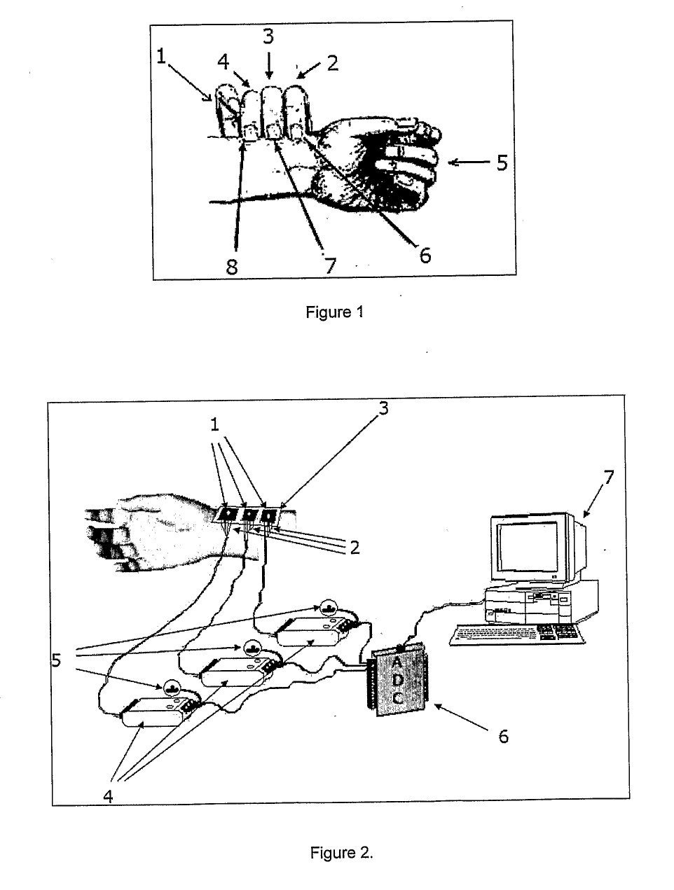

In the present invention, mounted over a neoprene sheet, 3 in FIG. 2, three pressure sensing elements, 1 in FIG. 2, coupled with transmitters, 4 in FIG. 2, which can amplify the electrical signal, are placed at the three predetermined locations, 6, 7, 8 in FIG. 1, in place of the fingertips of the Ayurvedic practitioner. The pressure sensing elements along with the neoprene sheet have to be properly adjusted on the patient's wrist considering the variable size of patient's wrist, skin differences, and such that all the three diaphragms, 2 in FIG. 4, of the three sensing elements exactly come in contact with the patient's nadi at the three predetermined locations on the wrist. The sensor leads, 2 in FIG. 2, are properly shielded. Each of the pressure sensing elements is supplied with the excitation voltage by using the DC power source, 5 in FIG. 2, through the transmitter. This arrangement generates an electrical signal proportional to the pressure experienced by the pressure sensing element, which is then digitized using the digitizer (ADC), 6 in FIG. 2, having an interface with the personal computer (PC), 7 in FIG. 2, at the USB port.

The data can be obtained on the computer for a predetermined length of time, for any change in the signal value, by using the data acquisition software, which controls the digitization as well. The minimum change in the signal, which can be measured, depends solely on the resolution of the digitizer.

The data obtained in this way is usually corrupted because of implicit and explicit electronic and electrical disturbances, called as noise, which modulates the information content. The noise level obtained in the present system developed is almost zero, after proper shielding. Hence the nadi obtained is in purer form and any digital filtering on the signal obtained from the digitizer, of any kind, is not required.

The waveforms obtained from the present invention contain typical physiological properties such as rhythm, self-similar nature, and chaotic nature. Rigorous machine learning algorithms are used to classify these waveforms, primarily defined in the Ayurvedic literature, as various types and sub-types of nadi patterns. The waveforms are accurate, complete, reproducible and noise-free to perform accurate diagnosis.

The methodology adapted involves:
(a) placement of each of the three pressure sensing elements at the exact pick-up points by the three fingertips (of Ayurvedic practitioner) respectively, where Nadi pulses are sensed and the analog pressure signal generated therein is digitized after removing the DC component;
(b) introducing an arrangement for an air gap between each of the sensors and the skin using a neoprene sheet with three holes;
(c) connecting at least up to one transmitter to each of the sensor which is further connected to the DC voltage supply from the other side;
(d) connecting at least one digitizer for converting the electrical signal as obtained from step (d) into digital form using at least one Analog to Digital Converter (ADC) for capturing the rapid changes in input signal, along with a shielding of filtering arrangement for minimizing the noise;
(e) recording and storing different parameters from the digital signals of primary and secondary peaks as obtained from step (d) into a storage device;
(f) designing dedicated programs in the storage device for optimizing a performance criterion of classification of pulse patterns;
(g) observing and interpreting the results obtained from above steps by analysis of pulse waveforms for detecting various disorders.

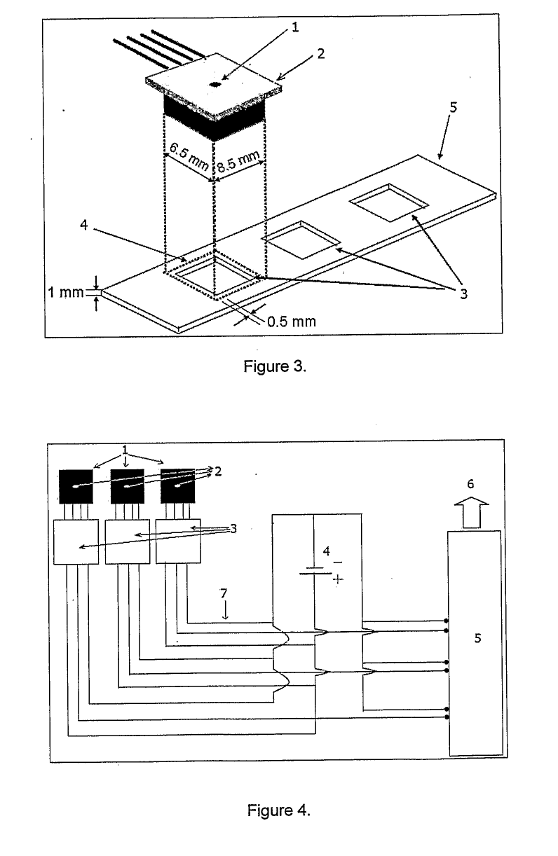

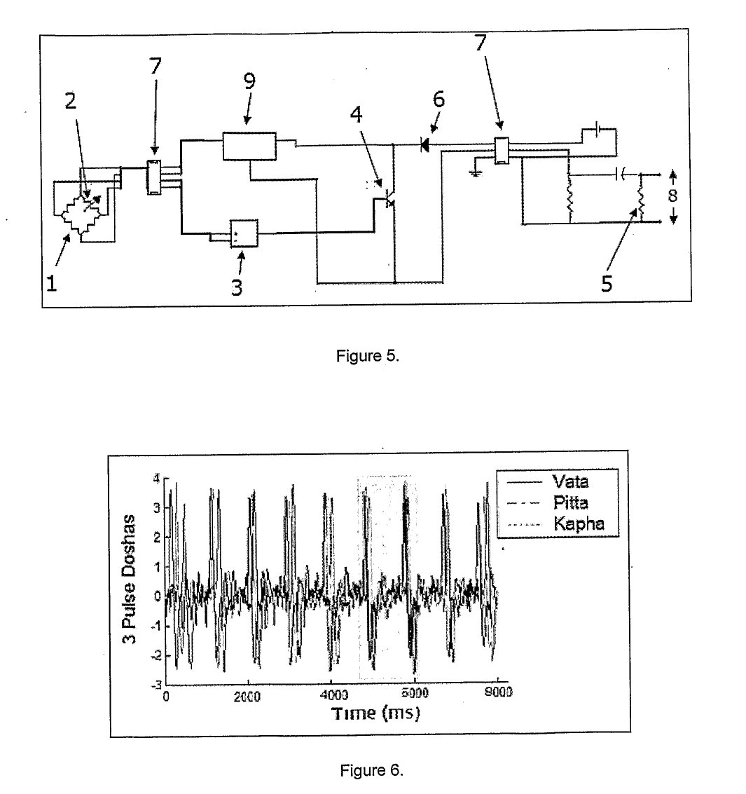

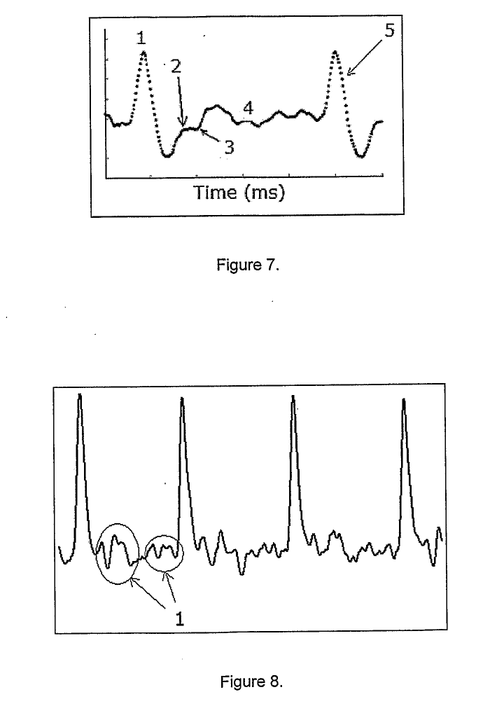

The detailed description of the system adapted is as follows.
FIG. 4 explains the electrical line diagram of the present invention. Each of the diaphragm, 2 in FIG. 4, based pressure sensing elements, 1 in FIG. 4, is supplied with the excitation voltage by using the DC power source, 4 in FIG. 4, through the transmitters, 3 in FIG. 4. Each output of the pressure sensing element is obtained from the transmitter through the corresponding connecting leads, 7 in FIG. 4. The output is further connected to the ADC, 5 in FIG. 4 for digitization and finally stored in computer, 6 in FIG. 4.
The details of the circuitry adapted for each sensing element in the present invention are disclosed in FIG. 5. The Wheatstone bridge, 1 in FIG. 5, of the pressure sensing element receives the constant excitation voltage from reference voltage generator, 9 in FIG. 5, through the connecting bus. The variable resistor, 2 in FIG. 5, of the bridge recognizes the pressure changes from the Nadi pulses. This output is amplified through a series of amplifiers, 3 in FIG. 5, and is given to the base of the NPN-type transistor, 4 in FIG. 5. The output is obtained from the emitter terminal, which is proportional to the amplified pressure signal from the bridge. The current output is converted into voltage, 8 in FIG. 5, by using a resistor, 5 in FIG. 5, which goes for digitization. The diode, 6 in FIG. 5, allows the unidirectional current flow. All the connecting wires, 7 in FIG. 5, were properly shielded and grounded which eliminate any external interference, noise.
FIG. 3 shows the arrangement of neoprene sheet, 5 in FIG. 3, to introduce air gap between sensors and the person's skin. The dimensions of each sensor are 8.5 mm×6.5 mm. A very tiny diaphragm, 1 in FIG. 3, is at the center of the sensor, 2 in FIG. 3, which has to be exactly placed at pre-defined position on wrist. Three holes, 3 in FIG. 3, are made into the neoprene sheet (of thickness 1 to 5 mm) for introducing air gaps, 4 in FIG. 3. The size of each hole is such that each sensor just rests on the sheet covering its respective hole.
Digitizer and data acquisition software: The analog signal obtained from the transmitter is freed from the DC component and is then subjected to the digitization by using an ADC. Bandwidth of the ADC is high enough to capture the rapid changes in the input signal from the transmitter. An ADC of accuracy 12-bit was used for our invention. The ADC is interfaced to the personal computer at the USB port. The software, LabVIEW, supports the abovementioned ADC device, which enables the operations of ADC through personal computer itself. The software acquires the digitized data of Nadi pulses for a prefixed time and saves the digitized pulse wave on the disk.
FIG. 6 gives a normalized sample pulse data from our database. The three colors indicate three different doshas captured at pre-defined positions on wrist. The three dosha waveforms almost follow each other, but they show different nature. The information hidden in these data are captured using various algorithms. FIG. 7 shows a zoomed version of a pulse cycle from FIG. 6 of one dosha, indicating the important time domain features. In our database, the details in Percussion wave, 1 in FIG. 7, Tidal wave, 2 in FIG. 7, Valley, 3 in FIG. 7, and Dicrotic wave, 4 in FIG. 7, show different behavior for different patients and thus can be identified by learning the behavior. Also the points-representation of pulse data, 5 in FIG. 7, gives the idea of the complete picture of pulse and that no extra information is available.

Hence the pulse time series, thus extracted consists of complete and noise-free spectra of the Nadi pulse. This is the unique feature of the present invention.

In an embodiment of the present invention, the parameters used are selected from the group comprising age, gender, profession, skin and atmospheric conditions.

In another embodiment of the present invention, the chaotic nature is determined in terms of strange attractor properties and the chaotic properties being captured in terms of Recurrence Quantification analysis parameters which are capable of capturing various disorders including fever, back-pain, arrhythmia and heart disorders.

In still another embodiment of the present invention, the variable resistor of the Wheatstone bridge is capable of recognizing the pressure changes at nadi pulses.

In yet another embodiment of the present invention, the device being capable of detecting arterial pulse pressure in the range of (−) 0.00124 Pa to (+) 0.00124 Pa.

In a further embodiment of the present invention, the type of nadi is selected from the group consisting of Sukshma, Tikshna, Kathina and Sama, their sub-types and combinations thereof, wherein the pressure points of the user are vata, pitta and kapha.

In another embodiment of the present invention, the pressure at the sensors is in the range of 7.5 to 13 cm H₂0 pressure for capturing accurate pressure readings.

In still another embodiment of the present invention, the thickness of neoprene sheet used is in the range of 1 to 5 mm.

In yet another embodiment of the present invention, the three sensing elements are mounted exactly on the three holes made [4 in FIG. 3] in a neoprene sheet with thickness in the range of 1 to 5 mm to introduce three air gaps between the sensors and the patient's skin so as to capture the tiny pressure very accurately.

In another embodiment of the present invention, the storage device is preferably a computer having at least one USB port.

In still another embodiment of the present invention, the waveform produced comprises domain features of percussion wave, tidal wave, valley and dicrotic wave.

In a further embodiment of the present invention, is provided a method for quantitative detection of arterial nadi pulse waveform of an individual using the claimed device Nadi Tarangini, wherein the said method comprising the steps of placing the said device at predetermined position for at least up to 60 seconds followed by acquiring and recording different parameters forming complete noiseless nadi waveform peaks characterized by typical physiological properties selected from the group comprising rhythm, self-similar nature, chaotic nature and then interpreting the results obtained for identifying possible disorders in a user.

In a further embodiment of the present invention, the sub-type of nadi is selected from the group consisting of Manda and Vegavati, wherein the pressure points of the user are vata, pitta and kapha.

In a further embodiment of the present invention, the pulse rate is quantitatively computed from the Fourier spectrum of the pulse.

In another embodiment of the present invention, the peaks include both main and secondary types and varies with change on different parameters.

In yet another embodiment of the present invention, the rhythm used is Pitch Synchronous Wavelet Transform, wherein the wavelet coefficients being capable of extracting the average values of the pulse to capture the essence of the whole data series.

In still another embodiment of the present invention, the self-similar nature of the waveform is determined by multifractal spectrum being capable of distinguishing various pulse patterns of different age groups of users.

In yet another embodiment of the present invention, the variations between consecutive pulse beats is captured by Pulse Variability, to capture the arrhythmic behavior present in the pulse.

In still another embodiment of the present invention, the chaotic properties in the pulse data are captured in terms of descriptor from Recurrence Plot to describe large and small-scale structures to detect disorders including fever.

### EXAMPLES

The following examples are given by way of illustration and therefore should not be construed to limit the scope of the present invention.

### Example 1

The Nadi pulses were recorded using our embodiment by placing the three pressure sensing elements, mounted on neoprene sheet, exactly at the three predetermined locations (6, 7, 8 in FIG. 1) on patient's left hand wrist, in place of the fingertips of the Ayurvedic practitioner. The three predetermined locations are vata position, pitta position and kapha position on the patient's wrist. The sampling rate of the acquisition was 500 Hz, which was enough to capture all the details. The data was collected for 1 to 5 minutes. All the three signals were individually digitized using the ADC (5 in FIG. 4) and were stored in the pulse database as vata pulse data, pitta pulse data and kapha pulse data respectively. Same procedure was followed for the patient's right hand wrist to get three more data. Therefore in the pulse database, 6 pulse signals (from vata, pitta and kapha positions on both the hands) were stored for each patient. Also the patient's information such as age, gender, profession was recorded in the database. The complete database contains information and pulse signals of 42 patients suffering from different disorders including fever, arrhythmic disorder. Each of the signals show variations in the parameters Amplitudes, Frequency, Rhythm, Depth and Power, and therefore carry different patterns with different information. We studied and analyzed all the pulse signals collectively using different machine learning algorithms to provide a non-invasive, easy-to-use and quick in response diagnostic device Nadi Tarangini, which eliminated all the human errors in the Nadi-Nidan performed manually by Ayurvedic practitioner for diagnostics. The important steps are briefly explained here (and shown in FIG. 1), and the details involved are given in the subsequent examples. Firstly, the Fourier coefficients are computed for a pulse signal of a patient (any one out of total 6 pulses, as the pulse rate is the same in all of them for the considered patients). The pulse rate is computed from the fundamental frequency in the Fourier spectrum. In order to check the reproducibility of our embodiment Nadi Tarangini, the pulse signals of a single person were recorded at different times in a morning session, and their correlation dimensions were computed to verify. As the length of each pulse signal is very high, we compute the average pulse values using the Pitch Synchronous Wavelet Transform to capture the essence of pulse. This averaged pulse can also further be used for the detection purpose. Using the above mentioned parameters and average pulse for all the 6 pulse signals for a patient, the four major types of Nadi (i.e. Sukshma, Tikshna, Kathina and Sama), the sub-types of Nadi (i.e. Manda and Vegavati) and their combinations were obtained. The detection was done using the classifier Support Vector Machine (SVM). Firstly, the classifier was trained using the parameters from first 31 patients and then tested for remaining 11 patients. Also, the pulses of patients showed different behavior prominently in three age-groups (i.e. “age below 25”, “age 25 to 50” and “age above 50”) and this behavior was captured using the multifractal analysis based on nonlinear dynamics and SVM. The arrhythmic behavior in pulse signal was captured using the variations in the pulse intervals using Pulse Rate Variability analysis and SVM. Finally, the chaos theory based Recurrence Plot analysis (based on recurrence quantification descriptors % recurrence, % determinism, entropy and % laminarity) was used to easily detect the disorders in the pulse signals using SVM.

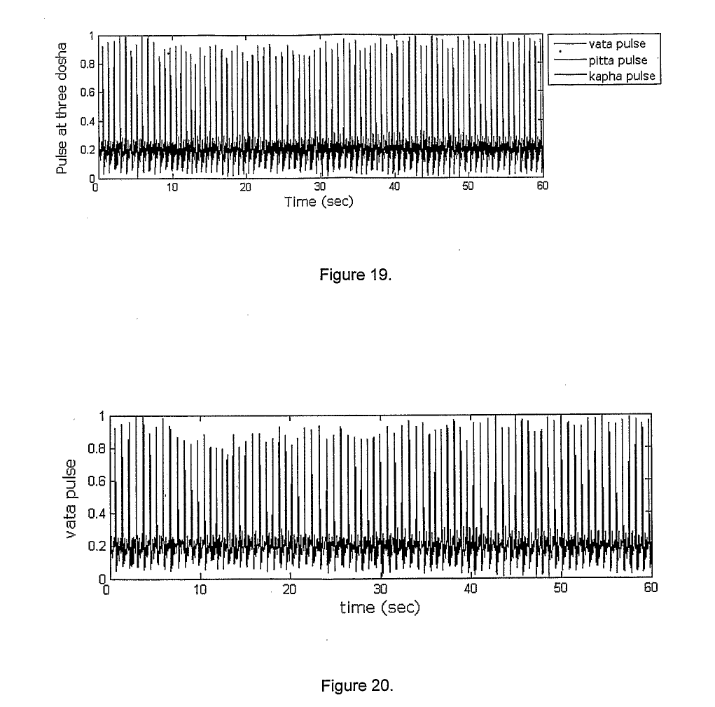

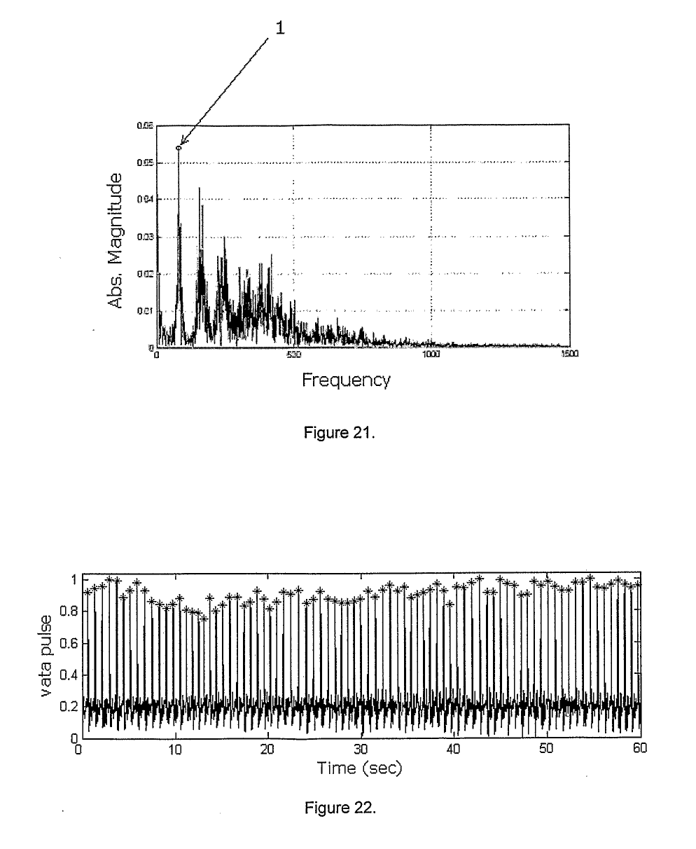

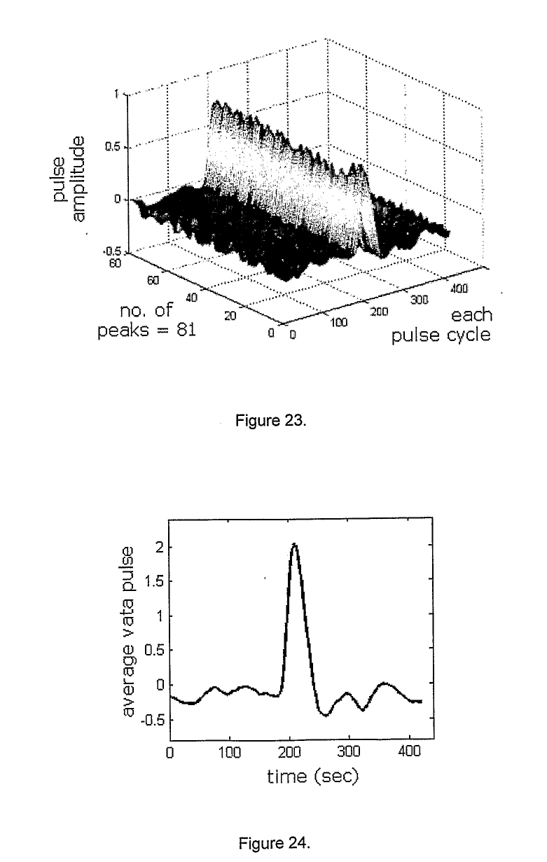

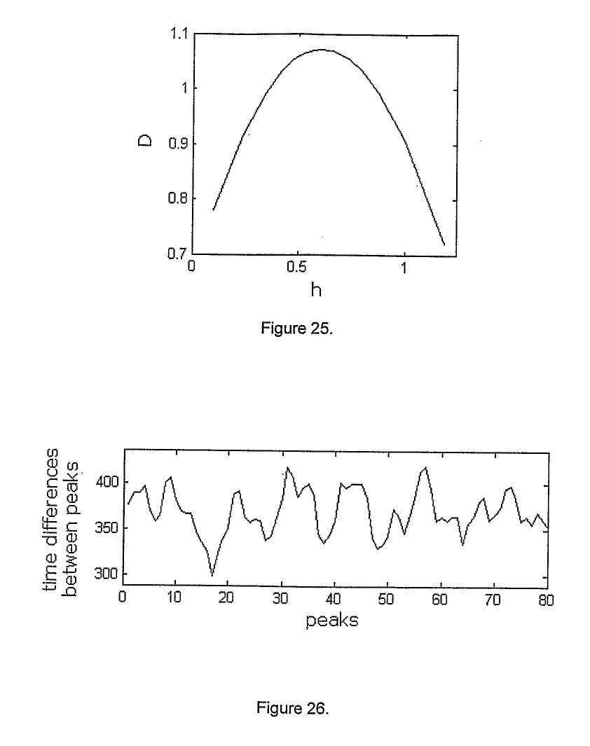

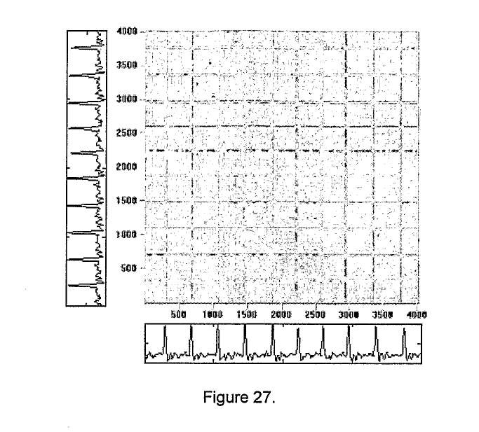

As an example, we show these steps and calculations for a sample pulse of person 34. FIG. 19 shows the complete pulse captured for 1 minute with sampling rate 500 Hz. Therefore, for the 3 doshas (at three predefined positions vata, pitta and kapha), the total no. of points are 3×60 (sec)×500 (Hz)=3×30,000=90,000. Only vata pulse is shown in the FIG. 20, which contains 30,000 points for 1 minute. Fourier Transform of vata pulse is computed, which gives 30,000 Fourier coefficients. Only the first 1500 coefficients (excluding the first one, which provides the average value) are plotted, for visibility, in FIG. 21. It can be noted that the first peak is at frequency 80.57 (=81), 1 in FIG. 21, which is the pulse rate of the person 34. The manually counted pulse rate is also 81. The correlation dimensions of the three doshas individually are 1.76, 1.71 and 1.75 respectively. For computing the average vata pulse, first the peaks in the vata pulse are computed as shown in FIG. 22, where the ‘red *’ points indicate peaks. Then the vata pulse is folded in such a manner that all the peaks are together as shown in FIG. 23. The wavelet transform of this folded vata pulse finally provides the average pulse as shown in FIG. 24. Also, it can be seen that the pulse movements are high, thus the sub-type of vata pulse is vegavati. The shapes at the Tidal and Dicrotic waves look like equilateral triangle, thus the vata pulse is also a Kathina pulse. Further, all the three doshas show equivalent behavior and thus the pulse is sama pulse. Then the multifractal analysis of vata pulse provides the multifractal spectrum as shown in FIG. 25, which captures the self-similarity. The peaks computed above are then used for pulse rate variability. In the considered vata pulse, there are 81 peaks, and thus 80 differences between them. These differences are all close enough as shown in FIG. 26, and thus the considered vata pulse is not arrhythmic. Finally the recurrence plot of vata pulse (only first 8,000 points out of 30,000 are shown for better visibility) in FIG. 27 shows the small- and large-scale structures in the vata pulse. The recurrence quantification descriptors using embedding dimension 7, time delay 1 and radius 0.3 are recurrence=5,579, laminarity=−2.182 and determinism=95. We finally used all the above results in the form of parameters for the diagnosis of person 34 by passing them to the classifier. The classifier SVM finally provides the outputs such as person 34 is of type sama kathina vegavati, person 34 does not have arrhythmic disorder.

### Example 2

Pulse rate: The pulses were obtained by placing the sensor at the predetermined position for 1 to 5 minutes. Immediately after the Nadi was taken, the pulse rate was measured manually for every acquisition. The pulse rate is computed using the fundamental frequency in the Fourier spectrum of any one dosha of the 6 pulse data of the patient. The comparison of pulse rate measured from a pulse time series and that manually measured for few of the patients is given in Table 2.

### Example 3

Reproducibility: The Nadi pulses were acquired of person 2 (age 27) at 7 different timings throughout a morning session (8:30 am, 9:15 am, 10:00 am, 10:45 am, 11.30 am, 12.15 pm and 1.10 pm) using our invention described in above description. Apart from the person's physic, Nadi is sensitive to mental status, stresses, thoughts, etc. Because of which the nature of the pulse essentially changes. For the above mentioned 7 timings, the person was asked to relax for 5 minutes before taking the pulse. Chaos analysis was carried on all the pulse data of the 7 timings, and it was observed that the Correlation Dimensions and Largest Lyapunov exponents [reference—D. Kugiumtzis, B. Lillekjendlie, and N. Christophersen. Chaotic time series part I: Estimation of some invariant properties in state space. Modeling, Identification and Control, 15(4):205-224, 1994] of the particular dosha remain almost constant, even though the shape of pulse changes slightly. The correlation dimensions of the pulses for vata, pitta and kapha of left hand are given in Table 3. Since the correlation dimensions (and largest Lyapunov exponents) throughout the morning session remained constant, it shows that the pulses obtained are completely reproducible, but the pulse shape may change slightly.

### Example 4

Computing essence of the pulse data: Each pulse data series is given to the Pitch Synchronous Wavelet Transform algorithm [reference—Evangelista, G. 1993. “Pitch Synchronous Wavelet Representations of Speech and Music Signals.” IEEE Transactions on Signal Processing 41(12):3313-3330] to extract the average values of the pulse, which capture the essence of the whole data series as shown in FIG. 10. The same procedure is carried for the other two dosha data series also. The Pitch Synchronous Wavelet Transform first finds the peaks in the time series, 1 in FIG. 10, folds the time series in such a manner that all the peaks come together, 2 in FIG. 10, and then takes the wavelet transform, 3 in FIG. 10, in z-direction, 4 in FIG. 10. The final outcome gives the average values throughout the pulse data series.

### Example 5

Identification of types of Nadi: The types of Nadi are identified using supervised classification. Firstly, various parameters such as Amplitudes, Frequency, Rhythm, Depth and Power are computed for all the pulse waveforms available in the database. The true Nadi types are also provided by the Ayurvedic practitioner in qualitative terms. Support Vector Machine (SVM) [reference—Vladimir N. Vapnik. The Nature of Statistical Learning Theory. Springer, New York, N.Y., USA, 1995] is used as the classifier. SVM rigorously based on statistical learning theory simultaneously minimizes the training and test errors, and produces a unique globally optimal solution. The parameters extracted from person 1 through person 31, along with their known Nadi types, are used for training the SVM. Then, the parameters of person 32 through person 42 are tested. The output labels of SVM (quantitatively determined labels using said method) are compared with the true Nadi types (qualitatively recorded labels from the database, provided by Ayurvedic practitioner). The comparison is given in Table 4. We could classify the pulses into the Nadi types as Sukshma, Sama, Kathina, Tikshna and their combinations with good accuracy.

### Example 6

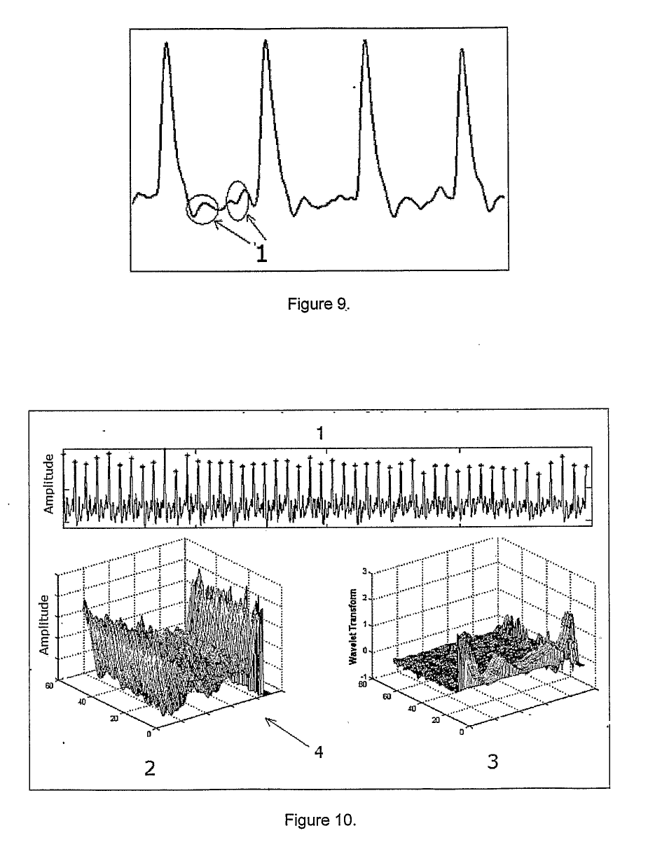

Identification of sub-types of Nadi: The pulse data are preliminary classified as Vegavati or Manda depending upon the pulse rate and the movement of the pulse. As shown in FIG. 8, if the pulse rate is very high, and the movement is higher, 1 in FIG. 8, then the pulse is detected as Vegavati pulse. On the other hand, as shown in FIG. 9, if the pulse rate is low with very less movements, 1 in FIG. 9, in Tidal and Dicrotic waves, then the pulse is detected as Manda pulse.

### Example 7

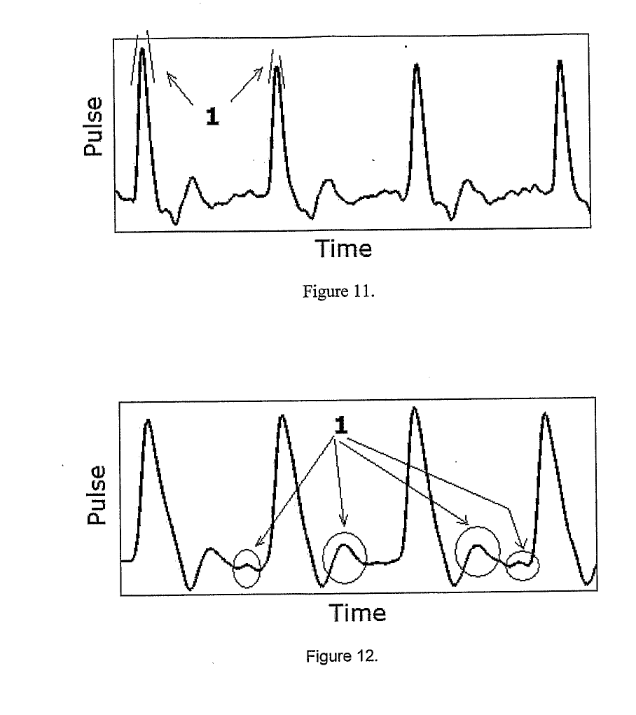

Identification of Tikshna Nadi: FIG. 11 shows vata pulse waveform of person 41 as an example of Tikshna Nadi, where the slopes at the peaks of Percussion wave are found to be very sharp, 1 in FIG. 11.

### Example 8

Identification of Kathina Nadi: FIG. 12 shows kapha pulse waveform of person 38 as an example of Kathina Nadi, where the shapes at the Tidal and Dicrotic waves look like equilateral triangle, 1 in FIG. 12.

### Example 9

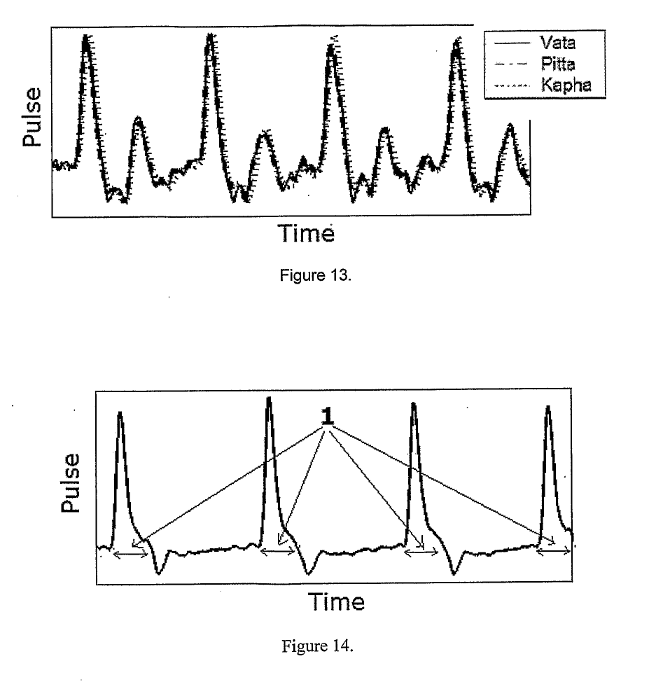

Identification of Sama Nadi: FIG. 13 shows all three pulse waveforms of person 40 as an example of Sama Nadi, where the pulse shows equivalent behaviour in all the three doshas.

### Example 10

Identification of Sukshma Nadi: FIG. 14 shows vata pulse of person 36 as an example of Kathina Nadi, where the pulse has very low slopes with wide widths of Tidal and Dicrotic waves, 1 in FIG. 14.

### Example 11

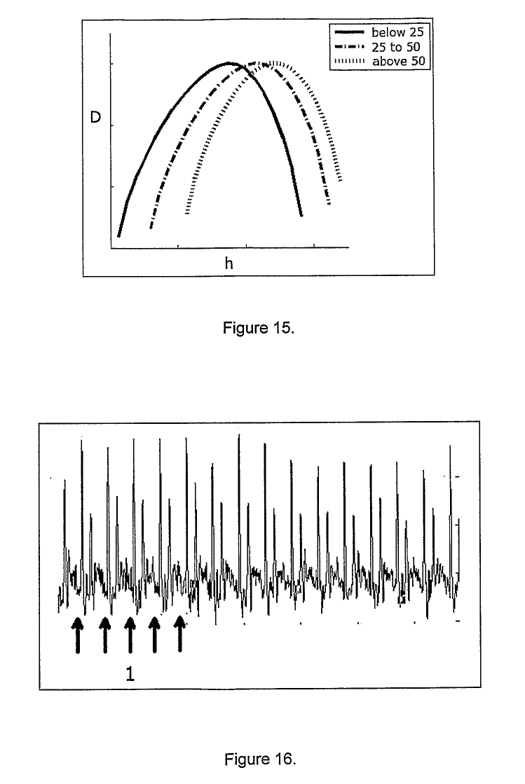

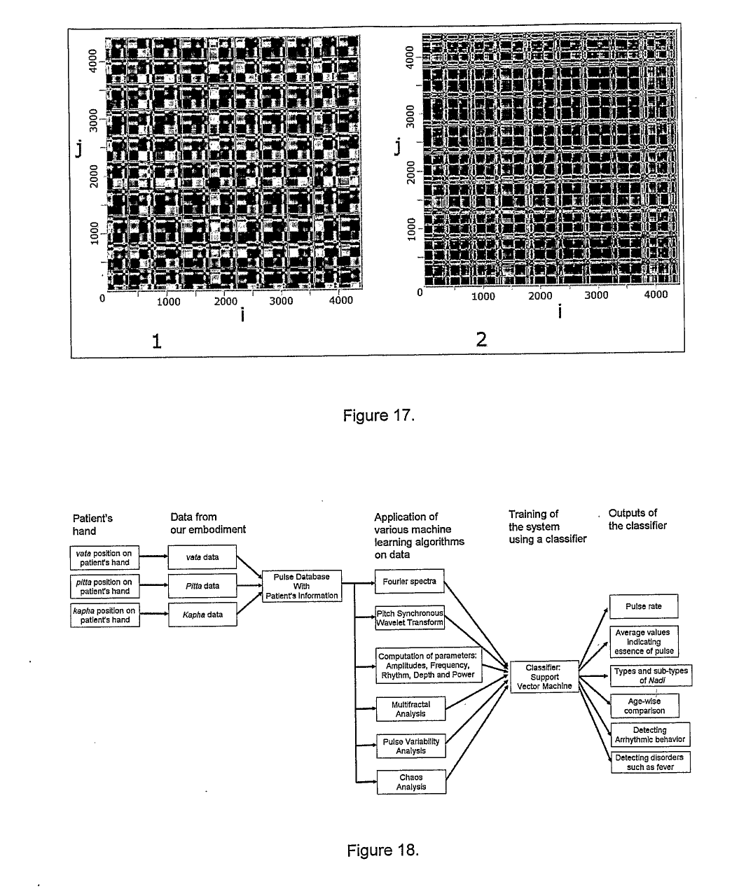

Identification of special pulses: Pulse Rate Variability, Multifractal spectrum analysis and Recurrence Plot methodologies are used for capturing the special cases of pulses in all the doshas.
A Multifractal spectrum [reference—J. F. Muzy, E. Bacry and A. Arneodo, The multifractal formalism revisited with wavelets. Int. J. Bif. Chaos 4 (1994) 245-302] captures the self-similarity of the pulse series, which is an essential property of a physiological time series. 22 normal pulses are separated into three age-groups namely “age below 25”, “age 25 to 50” and “age above 50” and their multifractal spectra are observed. In FIG. 15, multifractal spectrum of one randomly chosen normal pulse from each age-group is shown. As shown in FIG. 15, the multifractal spectrum moves towards top-up corner, as the age increases. Therefore, as explained in Example 5, a classifier can be trained to classify a pulse into once of the three age-groups.
Pulse variability [reference—L. Li and Z. Wang. Study on interval variability of arterial pulse. In The 1st Joint BMES/EMBS Conference, page 223, 1999] captures the variations between consecutive pulse beats, rather than simply the pulse rate. Firstly, the pulse peaks are detected and the difference between these peaks forms the pulse variability data. We use this pulse variability data to capture the missing pulse beats, if any, and thus the data is very useful to capture the arrhythmic behavior present in the pulse as shown in FIG. 16. In a normal pulse data, the differences between in pulse peaks vary in a very close range. In the considered pulse data, every third beat is missing, 1 in FIG. 16; therefore the differences between the peaks are varying and thus can be detected as an arrhythmic pulse data.
The chaotic properties in the pulse data can be captured in terms of Recurrence Plot (RP) [reference—J. P. Zbilut, C. L. Webber Jr.: Embeddings and delays as derived from quantification of recurrence plots, Physics Letters A, 171(3-4), 199-203 (1992)], whose quantification analysis describes large and small-scale structures through a set of descriptors. These descriptors are subsequently used to detect various disorders (e.g. fever) by training a classifier as explained in Example 5. FIG. 17 shows an example of recurrence plot of fever Pitta pulse, 2 in FIG. 17, which shows very different behavior than the recurrence plot of a normal pitta pulse, 1 in FIG. 17, and hence is identified using the descriptors.

### TABLES

Table 1: Characteristics of three humors (Vata, Pitta and Kapha) defined in Ayurveda.

Table 2: Comparison of the pulse rate.

Table 3: Comparison of the correlation dimensions (CD) of the pulses (from morning session) of person 2 for checking reproducibility.

Table 4: Identification of Nadi pulses using machine learning algorithms.

TABLE 1
Characteristics of three humors (Vata, Pitta and Kapha)
defined in Ayurveda.

| | VATA PULSE | PITTA PULSE | KAPHA PULSE |
|---|---|---|---|
| Characteristics | Fast, feeble, | Prominent, strong, | Deep, slow, broad |
| | cold, light, | high amplitude, | wavy, thick, cool, |
| | thin, disappears | hot, forceful, lifts | warm, regular |
| | on pressure | up the palpating | |
| | | fingers. | |
| Location | Best felt under | Best felt under | Best felt under |
| | the index finger | the middle | the ring |
| | | finger | finger |
| Gati | Moves like a | Moves like a frog | Moves like a |
| [Movement] | cobra | | swimming swan |

TABLE 2
Comparison of the pulse rate.

| | | PULSE RATE IN THE | |
|---|---|---|---|
| | | ACQUIRED DATA USING | PULSE RATE |
| AGE | | FOURIER ANALYSIS | MANUALLY |
| GROUP | PERSON | (rounded to nearest integer) | MEASURED |
| Below 25 | Person 1 | 68 | 69 |
| 25 to 50 | Person 2 | 78 | 76 |
| 25 to 50 | Person 3 | 79 | 78 |
| 25 to 50 | Person 4 | 75 | 75 |
| Above 50 | Person 5 | 82 | 82 |
| Above 50 | Person 6 | 111 | 112 |
| 25 to 50 | Person 7 | 74 | 78 |
| Above 50 | Person 8 | 81 | 81 |
| Below 25 | Person 9 | 68 | 69 |
| 25 to 50 | Person 10 | 82 | 80 |
| Below 25 | Person 11 | 66 | 66 |

TABLE 3
Comparison of the correlation dimensions (CD) of the pulses
(from morning session) of person 2 for checking reproducibility.

| | CD OF | CD OF | CD OF |
|---|---|---|---|
| TIMING | VATA PULSE | PITTA PULSE | KAPHA PULSE |
| 8.30 am | 1.4945 | 1.7466 | 1.5924 |
| 9.15 am | 1.5046 | 1.8286 | 1.6728 |
| 10.00 am | 1.4962 | 1.8649 | 1.6340 |
| 10.45 am | 1.5427 | 1.7723 | 1.6776 |
| 11.30 am | 1.5024 | 1.8343 | 1.6826 |
| 12.15 pm | 1.4667 | 1.8502 | 1.6785 |
| 1.10 pm | 1.5118 | 1.7363 | 1.7016 |

TABLE 4
Identification of Nadi pulses using machine learning algorithms

| | | | TRUE NADI LABEL |
|---|---|---|---|
| | | | PROVIDED BY |
| | | NADI LABEL OUTPUT | AYURVEDIC |
| SR. NO. | NAME | FROM SVM | PRACTITIONER |
| 1 | Person 32 | Sukshma Sama Kathina | Sama Kathina (Manda) |
| | | (Manda) | |
| 2 | Person 33 | Sukshma Sama | Sukshma Sama |
| 3 | Person 34 | Sama Kathina (Vegvati) | Sama Kathin (Vegvati) |
| 4 | Person 35 | Sukshma Kathina | Sukshma Kathina |
| 5 | Person 36 | Sukshma (Manda) | Sukshma (Manda) |
| 6 | Person 37 | Sukshma Sama | Sukshma Sama |
| 7 | Person 38 | Kathina (Vegvati) | Kathina (Vegvati) |
| 8 | Person 39 | Sama Kathina (Manda) | Kathina (Manda) |
| 9 | Person 40 | Sama (Manda) | Sama (Manda) |
| 10 | Person 41 | Tikshna | Tikshna |
| 11 | Person 42 | Sukshma Sama | Sukshma Sama |
| | | | (Vegavati) |

### ADVANTAGES:

1. Data Acquisition Methodology Using Air-Gap.
An air gap is introduced between each of the three sensors and the skin at wrist using a neoprene sheet with three holes. The dimensions of a sensor are 9×7 mm and the tiny diaphragm is at the center. The neoprene sheet is of thickness 1 to 5 mm. The three holes on this sheet which are of dimensions 7×5 cm are such that the sensors just fit around them. This arrangement helps to pick up the pressure exerted by the artery accurately.

2. Accurate, Complete Waveforms: Physiological Properties.
The waveforms obtained from our embodiment are accurate and complete (contain all the information), reproducible and thus contain the typical physiological properties such as rhythm, chaotic nature, self-similarity.

3. Pulse Patterns.
The waveforms obtained from our system show patterns which resemble the nadis defined in the Ayurvedic literature such as Sama, Kathina, Tikshna, Sukshma.

4. Diagnosis based on Ayuvedic Concepts.
Rigorous machine learning algorithms are applied to classify the pulse waveforms obtained from our system to diagnose a patient for various disorders and health parameters.

We claim:

1. A non-invasive device Nadi Tarangini, useful for quantitative detection of arterial ‘nadi’ pulse waveform, wherein the said assembly comprising:
[a] at least one strip of neoprene [5 in FIG. 3] provided with at least three holes at three predetermined points [6, 7, 8 in FIG. 1];
[b] at least three circuits of diaphragm based pressure sensors [1 in FIG. 2] placed side by side at said at least three predetermined points on said strip of neoprene for sensing the ‘nadi’ pulses;
[c] said holes [3 in FIG. 3] having size smaller than the sensors such that each sensor just rests on the sheet covering its respective hole so as to introduce air gaps between sensors and the patient's skin having thickness in the range of 1 to 5 mm for capturing the arterial pulsations;
[d] providing at least one transducer [1 in FIG. 2] coupled with each of the said pressure sensor provided above along with a DC power source [4 in FIG. 4] for converting the pressure signal into an equivalent electrical signal;
[e] connecting at least one digitizer [5 in FIG. 4] for converting the electrical signal obtained in step [d] above into digital form, using at least one Analog to Digital Converter (ADC) [5 in FIG. 4], along with a shielding arrangement [7 in FIG. 5] for minimizing the noise;
[f] providing a computing device [7 in FIG. 2] connected to the said digitizer for obtaining the visual display of the pulse pressure waveform.

2. A device according to claim 1, wherein the circuit of the diaphragm based pressure sensor comprising:
i. wheatstone bridge [1 in FIG. 5] for receiving the constant excitation voltage from reference voltage generator [9 in FIG. 5] through the connecting bus [7 in FIG. 5];
ii. amplifiers corresponding to the numbers of transducers used [1 in FIG. 4] for amplifying the output;
iii. a base [4 in FIG. 5] of the NPN-type transistor,
iv. an emitter terminal [8 in FIG. 5] proportional to the amplified pressure signal from the bridge for obtaining the output;
v. a diode or resistor [6 in FIG. 5] allowing unidirectional current flow for converting current output into voltage, which goes for digitization;
vi. connecting wires [7 in FIG. 5] being property shielded and grounded to eliminate external interference and noise.

3. A device according to claim 1, wherein the variable resistor of the Wheatstone bridge is capable of recognizing the pressure changes at ‘nadi’ pulses.

4. A device according to claim 1, being capable of detecting arterial pulse pressure in the range of (−) 0.00124 Pa to (+) 0.00124 Pa.

5. A device according to claim 1, wherein the pressure at the sensors is in the range of 7.5 to 13 cm H₂O pressure for capturing accurate pressure readings.

6. A device according to claim 1, wherein the three sensing elements are mounted exactly on the three holes made [4 in FIG. 3] in a neoprene sheet to Introduce three air gaps between the three sensors and the patient's skin so as to capture the tiny pressure very accurately at the three predetermined locations on wrist.

7. A device according to claim 1, wherein the thickness of neoprene sheet used is in the range of 1 to 5 mm.

8. A device according to claim 1, wherein the computing device is preferably a computer having storage and at least one USB port.

9. A device according to claim 1, wherein the waveform produced comprises domain features of percussion wave, tidal wave, valley and dicrotic wave.

10. A method for quantitative detection of arterial nadi pulse waveform of an individual, using the device Nadi Tarangini according to claim 1, wherein the said method comprises the steps of capturing the arterial pulse, pressure with the help of diaphragm based pressure, sensors mounted on the neoprene strip with three holes to introduce air gaps, by placing the said device at predetermined position for at least up to 60 seconds followed by converting the pressure signal in to electrical signal with the help of transducers and then in to digital form with the help of analog to digital converter, acquiring and recording thus obtained data forming complete noiseless nadi waveform, characterized by typical physiological properties selected from the group comprising pulse rate, self-similar nature, chaotic nature, average pulse behavior and identifying the possible disorders in a user.

11. A method according to claim 10, wherein the type of nadi is selected from the group consisting of Sukshma, Tikshna, Kathina and Sama, and their combinations thereof, wherein the pressure points of the user are vats, pitta and kapha.

12. A method according to claim 10, wherein the sub-type of nadi is selected from the group consisting of Manda and Vegavati, wherein the pressure points of the user are vata, pitta and kapha.
then interpreting the results obtained for identifying the types and sub-types of nadi and also

13. A method according to claim 10, wherein the peaks include both main and secondary types and vary with the changes on different parameters.

14. A method according to claim 10, wherein the pulse rate is quantitatively computed from the Fourier spectrum of the pulse.

15. A method according to claim 10, wherein the average pulse behavior is captured using Pitch Synchronous Wavelet Transform, wherein the wavelet coefficients being capable of extracting the average values of the pulse to capture the essence of the whole data series.

16. A method according to claim 10, wherein the self-similar nature of the waveform is determined by multifractal spectrum being capable of distinguishing various pulse patterns of different age groups of users.

17. A method according to claim 10, wherein the variations between consecutive pulse beats are captured by Pulse Rate Variability, to capture the arrhythmic behavior present in the pulse.

18. A method according to claim 10, wherein the chaotic properties in the pulse data are captured in terms of descriptors from Recurrence Plot to describe large and small-scale structures to detect disorders including fever.
